(19) United States
(12) Patent Application Publication
(10) Pub. No.: US 2013/0172838 A1
(43) Pub. Date: Jul. 4, 2013

(54) Negative Pressure and Electrostimulation Therapy Apparatus

(71) Applicant: Kathleen Tremblay, Westfield, MA (US)

(72) Inventor: Kathleen Tremblay, Westfield, MA (US)

(73) Assignee: TYCO HEALTHCARE GROUP LP, Mansfield, MA (US)

(21) Appl. No.: 13/609,772

(22) Filed: Sep. 11, 2012

Related U.S. Application Data
(63) Continuation of application No. 12/510,694, filed on Jul. 28, 2009, now abandoned.
(60) Provisional application No. 61/151,316, filed on Feb. 10, 2009.

(51) Int. Cl. A61N 1/36; A61M 1/00

(52) U.S. Cl. CPC A61N 1/36014; A61M 1/0023
USPC 604/319

(57) ABSTRACT

A negative wound pressure therapy apparatus includes a wound dressing for defining a reservoir over a wound in which a negative pressure may be maintained by forming a substantially fluid-tight seal around wound tissue. The apparatus also includes a fluid conduit in fluid communication with the reservoir. The fluid conduit defines a fluid flow path for carrying fluids from the reservoir. The apparatus also includes a vacuum source in fluid communication with the fluid conduit. The vacuum source is suitable for providing an appropriate negative pressure to the reservoir to stimulate healing of the wound. The apparatus also includes at least one biomedical electrode mounted with respect to the wound dressing for transmitting electrical energy to stimulate healing of the wound tissue.

### CROSS-REFERENCE TO RELATED DOCUMENTS

The present invention claims the benefit of and priority to U.S. provisional patent Application Ser. No. 61/151,316, filed on Feb. 10, 2009, disclosure of which may be referred to herein by reference.

### BACKGROUND

1. Technical Field

The present disclosure relates generally to a wound therapy apparatus. In particular, the disclosure relates to a wound therapy apparatus incorporating negative pressure wound therapy and electrostimulation therapy for use in promoting wound healing.

2. Background of Related Art

One technique that has proven effective in promoting the healing of wounds is known as negative wound pressure therapy (NPWT). Application of a negative pressure, e.g. reduced or sub-atmospheric pressure, to a localized reservoir over a wound has been found to assist in closing the wound by promoting blood flow to the area, stimulating the formation of granulation tissue and encouraging the migration of healthy tissue over the wound. A negative pressure may also inhibit bacterial growth by drawing fluids from the wound such as exudates, which may tend to harbor bacteria. This technique has proven particularly effective for chronic or healing-resistant wounds, and is also used for other purposes such as post-operative wound care.

The general NPWT protocol provides for a wound to be covered to facilitate suction at the wound area. For example, a flexible membrane having an adhesive periphery might be used to form a substantially fluid-tight seal around a perimeter of the wound, thus providing a reservoir over the wound where a negative pressure may be maintained. A fluid conduit may include a vacuum tube introduced into the reservoir through the membrane to provide fluid communication to an external vacuum source. Atmospheric gas, wound exudates or other fluids may thus be drawn from the reservoir through the fluid conduit to stimulate healing of the wound. Exudates drawn from the reservoir may be deposited in a collection canister until the canister may be conveniently emptied or replaced.

Another technique that has proven effective in promoting the healing of wounds is known as electrotherapy or electrostiumulation. The technique consists generally of applying two electrodes to the skin of the patient and passing an electric current between the electrodes so that the current enters a wound. The current promotes wound healing by increasing capillary density and perfusion and improving wound oxygenation.

Accordingly, a device for therapeutic treatment of wounds that incorporates an NPWT apparatus and an electrostimulation apparatus would maximize the capacity to evacuate exudate from a wound while further promoting healing of the wound using electric current.

### SUMMARY

The present disclosure describes a negative wound pressure therapy apparatus including a wound dressing for defining a reservoir over a wound in which a negative pressure may be maintained by forming a substantially fluid-tight seal around wound tissue. The apparatus also includes a fluid conduit in fluid communication with the reservoir. The fluid conduit defines a fluid flow path for carrying fluids from the reservoir. The apparatus also includes a vacuum source in fluid communication with the fluid conduit. The vacuum source is suitable for providing an appropriate negative pressure to the reservoir to stimulate healing of the wound. The apparatus also includes at least one biomedical electrode mounted with respect to the wound dressing for transmitting electrical energy to stimulate healing of the wound tissue.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a schematic diagram of an embodiment of an NPWT apparatus including a fluid conduit coupled with a wound dressing in accordance with the present disclosure;

FIG. 2A is a top view of an embodiment of the wound dressing of FIG. 1 having a pair of electrodes separated by an insulative material in accordance with the present disclosure;

FIG. 2B is a perspective view of the fluid conduit of FIG. 1 disengaged from the wound dressing of FIG. 2A;

FIG. 3A is a schematic diagram of an embodiment of an NPWT apparatus including a fluid conduit disengaged from a wound dressing in accordance with the present disclosure; and

FIG. 3B is a perspective view of a cover layer of the wound dressing of FIG. 3A.

### DETAILED DESCRIPTION

The wound therapy apparatus of the present disclosure promotes healing of a wound by providing a reservoir over the wound where a reduced pressure may be maintained. The reservoir subjects the wound to a negative pressure to effectively draw wound fluid, including liquid exudates, from the wound without the continuous use of a vacuum pump. Hence, negative pressure may be applied once, or may be varied depending on the nature and severity of the wound. A pair of electrodes incorporated within the apparatus provides electrostimulation therapy to the wound by running electric current from one electrode through the wound to the other electrode to accelerate healing of the wound. The attached figures illustrate exemplary embodiments of the present disclosure and are referenced to describe the embodiments depicted therein. Hereinafter, the disclosure will be described in detail by explaining the figures wherein like reference numerals represent like parts throughout the several views.

The wound therapy system of the present disclosure promotes healing of a wound via the use of a wound dressing and subatmospheric pressure mechanism. Generally, the subatmospheric pressure mechanism applies subatmospheric pressure to the wound to effectively remove wound fluids or exudate captured within the boundary of the composite wound dressing, and to increase blood flow to the wound bed and enhance cellular stimulation of epithelial and subcutaneous tissue. The wound therapy system may be entirely portable, i.e., it may be worn or carried by the subject such that the subject may be completely ambulatory during the therapy period. The wound therapy system including the subatmospheric pressure mechanism and components thereof may be entirely reusable or may be entirely disposable after a predetermined period of use or may be individually disposable whereby some of the components are reused for a subsequent therapy application.

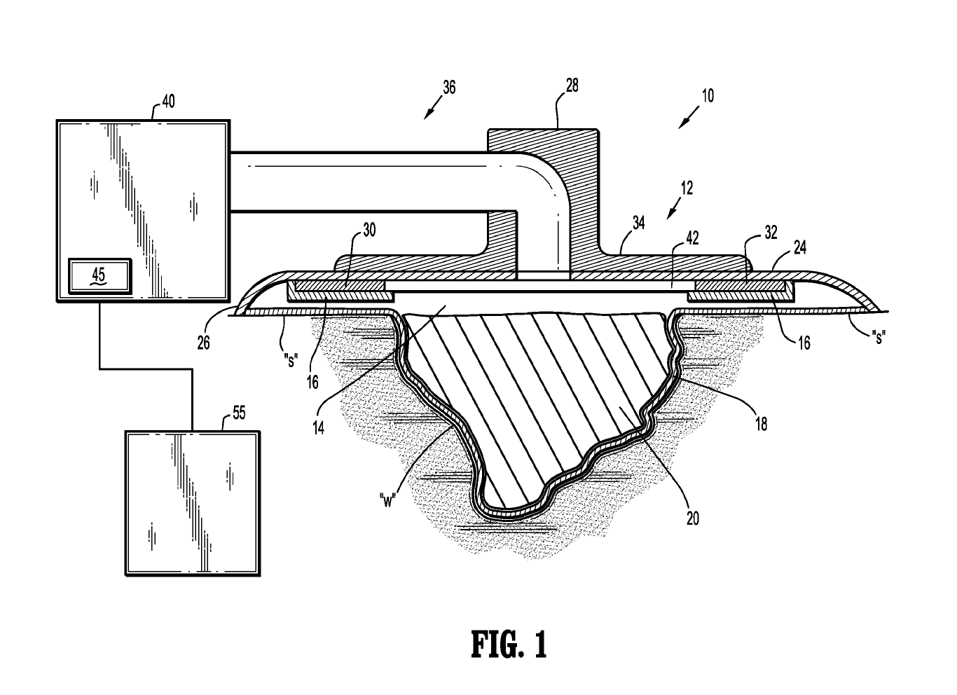

Referring initially to FIG. 1, an NPWT apparatus according to the present disclosure is depicted generally as 10 for use on a wound “w” surrounded by healthy skin “s.” The NPWT apparatus 10 includes a wound dressing 12 positioned relative to the wound “w” to define a reservoir 14 in which a negative pressure appropriate to stimulate healing may be maintained.

Wound dressing 12 includes a contact layer 18 positioned in direct contact with the bed of wound “w” and may be formed from perforated film material. An appropriate perforated material permits the negative pressure applied to the reservoir to penetrate into the wound “w,” and also permits exudates to be drawn through the contact layer 18. Passage of wound fluid through the contact layer 18 is preferably unidirectional such that exudates do not flow back into the wound bed. Unidirectional flow may be encouraged by directional apertures formed in the contact layer 18, or a lamination of materials having absorption properties differing from those of contact layer 18. A non-adherent material may be selected such that contact layer 18 does not tend to cling to the wound “w” or surrounding tissue when it is removed. One exemplary material that may be used as a contact layer 18 is sold under the trademark XEROFLO® by Tyco Healthcare Group LP (d/b/a Covidien). Another example of a material that may be suitable for use as the contact member 18 is the commercially available CURITY® non-adherent dressing offered by Tyco Healthcare Group LP (d/b/a Covidien).

Wound filler 20 is positioned in the wound “w” over the contact layer 18 and is intended to allow wound dressing 12 to capture wound exudates and transport these fluids through the dressing 12. Wound filler 20 is conformable to assume the shape of any wound “w” and may be packed up to the level of healthy skin “s.” The filler may be treated with agents such as polyhexamethylene biguanide (PHMB) to decrease the incidence of infection, or other medicaments to promote healing of the wound. A suitable wound filler 20 is the antimicrobial dressing sold under the trademark KERLIX™ AMD offered by Tyco Healthcare Group LP (d/b/a Covidien). The wound filler 20 may be saturated with saline or other conductive fluid to facilitate dispensing of electrical energy.

Wound dressing 12 also includes a cover layer 24 in the form of a flexible membrane. Cover layer 24 may be positioned over the wound “w” such that a biocompatible adhesive at the periphery 26 of the cover layer 24 forms a substantially fluid-tight seal with the surrounding skin “s.” Thus, cover layer 24 may act as both a microbial barrier to prevent contaminants from entering the wound “w,” and also a fluid barrier maintaining the integrity of vacuum reservoir 14. Cover layer 24 is preferably formed from a moisture vapor permeable membrane to promote the exchange of oxygen and moisture between the wound “w” and the atmosphere. A membrane that provides a sufficient moisture vapor transmission rate (MVTR) is a transparent membrane sold under the trade name POLYSKIN®II offered by Tyco Healthcare Group LP (d/b/a Covidien). A transparent membrane permits an assessment of wound conditions to be made without requiring removal of the cover layer 24. Alternatively, cover layer 24 may comprise an impermeable membrane or a substantially rigid member.

A vacuum port 28 may also be included in wound dressing 12 to facilitate connection of the wound dressing 12 to other apparatus components. The vacuum port 28 may be configured as a rigid or flexible, low-profile component having a hollow interior in fluid communication with the reservoir 14. An adhesive on the underside of a flange 34 may provide a mechanism for affixing the vacuum port 28 to the dressing 12, or alternatively flange 34 may be positioned within reservoir 14 (not shown) such that an adhesive on an upper side of the flange 34 affixes the vacuum port 28. Vacuum port 28 may be adapted to receive a fluid conduit 36 in a releasable and fluid-tight manner to provide fluid communication between the fluid conduit 36 and the reservoir 14. Fluid conduit 36 defines a flow path though the apparatus 10 for fluids such as wound exudates and atmospheric gasses. Vacuum port 28 may be eliminated from dressing 12 if other provisions are made for providing fluid communication with the fluid conduit 36.

Fluid conduit 36 connects wound dressing 12 to a vacuum source 40 that generates or otherwise provides a negative pressure to the NPWT apparatus 10. Vacuum source 40 may comprise a peristaltic pump, a diaphragmatic pump or other mechanism that is biocompatible and draws fluids, e.g. atmospheric gasses and wound exudates, from the reservoir 14 appropriate to stimulate healing of the wound “w.” Preferably, the vacuum source 40 is adapted to produce a sub-atmospheric pressure in the reservoir 14 ranging between about 20 mmHg and about 500 mmHg, more preferably, about 75 mmHg to about 125 mmHg, or even more preferably between about 30 mmHg to about 75 mmHg. One suitable peristaltic pump is the KANGAROO PET™ Enteral Feeding Pump manufactured by Tyco Healthcare Group (d/b/a Covidien).

Wound dressing 12 also includes at least one or a pair of electrodes 30 and 32 to facilitate the flow of current from one electrode through wound “w” to the other electrode to accelerate healing of wound “w”. As shown in FIG. 1, electrodes 30 and 32 may be disposed on skin “s” adjacent wound “w” (e.g., on opposing sides of wound “w”). In this scenario, current flows through the wound from one electrode positioned outside the wound to the other electrode positioned outside the wound. Electrodes 30 and 32 may be adapted to electrically connect to a suitable power supply 45 to conduct electrical energy from the power supply 45 to the wound. In embodiments, power supply 45 produces direct current. In other embodiments, power supply 45 produces an alternating electrical current. The current of choice is contingent upon the nature and severity of the injury. In the illustrated embodiment, power supply 45 is shown incorporated within vacuum source 40 (e.g., in a so-called “onboard” configuration), however, it should be understood that power supply 45 may be configured to operate independent of vacuum source 40. In embodiments, electrodes 30, 32 may be configured to operate wirelessly. More specifically, electrodes 30, 32 may include a local power supply (e.g., a battery) disposed thereon to provide electrical energy. Electrodes 30 and 32 may be made from materials that include aluminum, copper, Mylar™, metalized Mylar™, silver, gold, stainless steel or other suitable conductive material and may be of various shapes and may be arranged in various configurations and orientations. Electrodes 30 and 32 are separated by an insulative material 42 to prevent or greatly reduce the flow of current between electrodes 30 and 32 within dressing 12. Insulative material 42 may be composed of any high resistance material such as polythylene, poly(tetrafluoroethylene) (TEFLON™), polyurethane, polyester, a hydrogel made to be an insulator or any other suitable insulative material. To maintain electrodes 30, 32 and insulative material 42 within wound dressing 12, an adhesive on the underside of cover layer 24 or on the upper side of electrodes 30, 32 and insulative material 42 may provide a mechanism for affixing the electrodes 30, 32 and insulative material 42 to the cover layer 24.

In alternative embodiments, the NPWT apparatus 10 may incorporate at least one electrode (e.g., electrode 30), referred to as the active electrode, disposed within wound dressing 12 and in contact with the wound “w”. Another electrode 55, referred to as the return electrode, may be interfaced with a suitable location of the patient's skin (e.g., underneath the patient, adjacent the wound “w”, etc.). Electrical energy is supplied to the wound “w” by power supply 45 via a supply line (not shown) that is connected to the power supply 45, allowing the active electrode (e.g., electrode 30) to conduct the electrical energy through the wound “w” before returning to the power supply 45 through the return electrode 55 via a return line (not shown).

Wound dressing 12 also includes a conductive adhesive layer 16 contacting the underside of electrodes 30, 32 and insulative material 42. Conductive adhesive layer 16 may include gaps or spaces (not shown) between electrodes 30 and 32 (e.g., along the underside of insulative material 42) sufficient to prevent short circuiting. Alternatively, conductive adhesive layer 16 may only be applied to electrodes 30, 32. Conductive layer 16 may be a hydrogel, fibrin, or other suitable electrically conductive material capable of conducting electrical current through skin surfaces. Conductive layer 16 may include antimicrobial agents, antiseptic agents, vitamin E, or other agents for promoting wound healing.

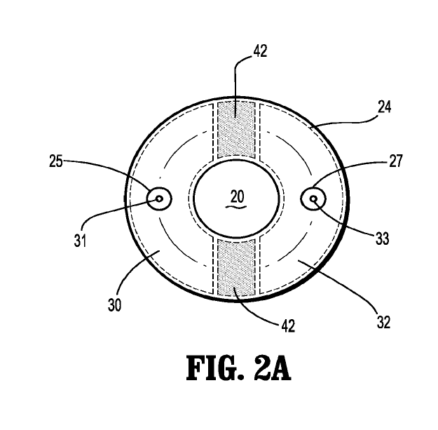

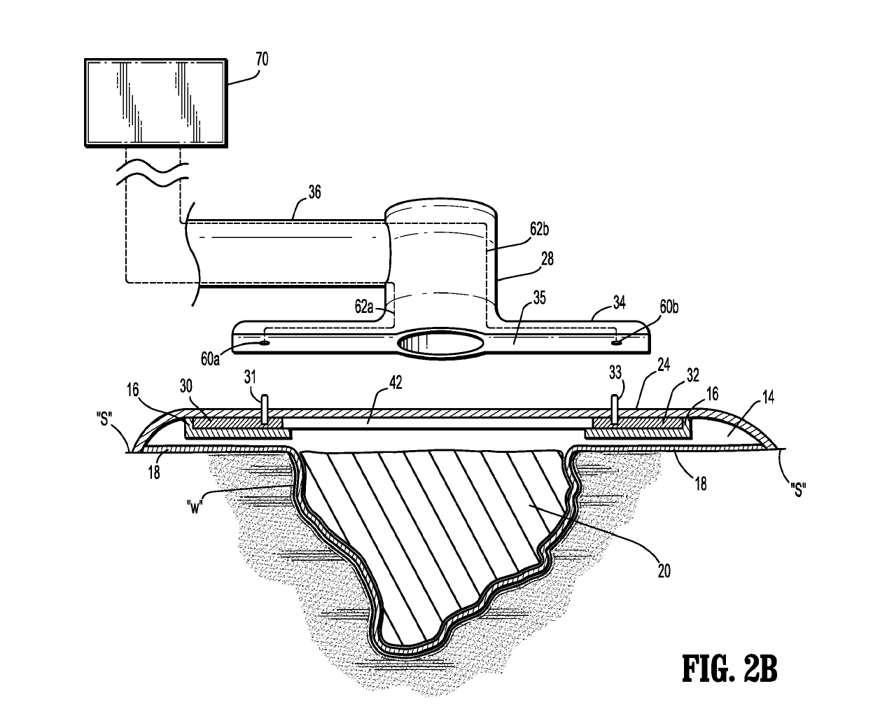

With reference to FIGS. 2A and 2B, cover layer 24 and conductive layer 16, may be generally ring-like in shape to define a flow path through wound dressing 12 from wound “w” to fluid conduit 36. Further, electrodes 30 and 32 may be generally arcuate in shape and are separated at each end by insulative material 42, such that electrodes 30, 32 and insulative material 42 together form a generally ring-like shape surrounding flow path 50. The generally ring-like shape configuration of wound dressing 12 allows for unimpeded fluid communication between the fluid conduit 36 and reservoir 14 via the flow path 50. Alternatively, cover layer 24 may be perforated (not shown) to accommodate the fluid conduit 36 within flow path 50 to provide fluid communication between the fluid conduit 36 and the reservoir 14. Each electrode 30 and 32 includes an electrical contact 31 and 33, respectively, that is positioned to pass through a pair of corresponding openings 25 and 27 in cover layer 24.

The wound filler 20 may be saturated with saline or other conductive fluid to facilitate dispensing of electrical energy from electrodes 30, 32 through conductive layer 16 to the wound “w”.

Flange 34 includes a pair of connectors 60a and 60b (e.g., snap connectors) adapted to electrically connect to a suitable power source 70 via electrical conductors 62a and 62b (shown in phantom). Upon affixing flange 34 to wound dressing 12, connectors 60a and 60b align with and receive electrical contacts 31 and 33 (e.g., in a snap-fit manner) to place electrodes 30 and 32 in electrical communication with connectors 60a and 60b, respectively. A biomedical electrode connector for coupling with a biomedical electrode of the type including an electrode base and a male terminal projecting from the electrode base is described in Provisional Application No. 61/012,817, filed on Dec. 11, 2007, the disclosure of which is incorporated herein by reference in its entirety.

One or more lumens (not shown) may be defined longitudinally through fluid conduit 36 to support conductors 62a and 62b within fluid conduit 36 for connection between connectors 60a and 60b and power source 70. In the illustrated embodiment of FIG. 2B, fluid conduit 36 and flange 34 are integrally formed as a monolithic structure (e.g., manufactured using a molding plate). The monolithic configuration allows a clinician to secure the fluid conduit 36 in place with the wound dressing 12 using connectors 60a, 60b and contacts 31, 33, such that the clinician is free to operate the vacuum source 40. Upon securing fluid conduit 36 in place using connectors 60a, 60b and contacts 31, 33, a biasing force between a distal end of fluid conduit 36 and cover layer 24 and/or between an inner face 35 of flange and cover layer 24 operates to form a fluid-tight seal between fluid conduit 36 and cover layer 24. Additionally or alternatively, an O-ring seal may be fitted on a distal end of fluid conduit 36 or be disposed on cover layer 24 to facilitate a fluid-tight seal between fluid conduit 36 and cover layer 24.

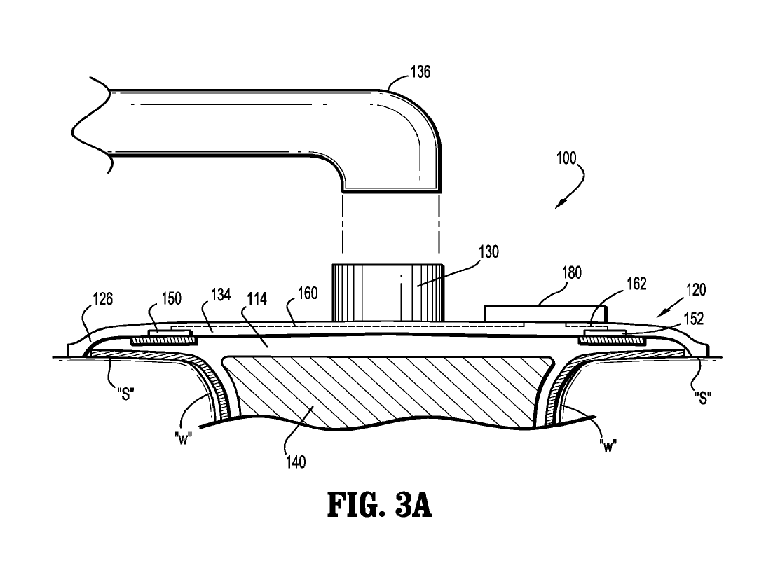

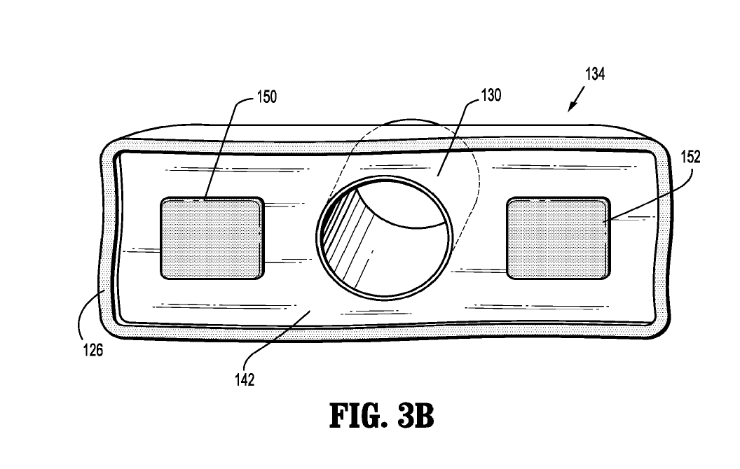

FIGS. 3A and 3B illustrate another embodiment of the presently disclosed NPWT apparatus shown generally as 100. NPWT apparatus 100 is substantially as described above with respect to apparatus 10 and will only be described to the extent necessary to explain its difference.

The NPWT apparatus 100 includes a wound dressing 120 positioned relative to the wound “w” to define a reservoir 114 in which a negative pressure appropriate to stimulate healing may be maintained. Wound dressing 120 includes a contact layer 118 positioned in direct contact with the bed of wound “w”. Unidirectional flow may be encouraged by directional apertures formed in the contact layer 118.

Wound filler 140 is positioned in the wound “w” over the contact layer 118 and is intended to allow wound dressing 120 to capture wound exudates and transport these fluids through the dressing 120.

Wound dressing 120 also includes a cover layer 134 in the form of a flexible flange (similar to flange 34 of FIG. 1) adapted to connect the wound dressing 120 to other apparatus components. Cover layer 134 includes a vacuum port 130 to facilitate connection of the wound dressing 120 to a fluid conduit 136. More specifically, vacuum port 130 is adapted to receive fluid conduit 136 (e.g., via a bayonet-type coupling) in a releasable and fluid-tight manner to provide fluid communication between the fluid conduit 136 and the reservoir 114. Fluid conduit 136 defines a flow path though the apparatus 100 for fluids such as wound exudates and atmospheric gasses. The vacuum port 130 may be configured as a rigid or flexible, low-profile component having a hollow interior in fluid communication with the reservoir 114. Cover layer 134 may be positioned over the wound “w” such that a biocompatible adhesive at the periphery 126 of the cover layer 134 forms a substantially fluid-tight seal with the surrounding skin “s.”

Wound dressing 120 also includes a pair of electrodes 150 and 152 disposed between the cover layer 134 and the contact layer 118 to facilitate the flow of current from one electrode through wound “w” to the other electrode to accelerate healing of wound “w”. As shown in FIG. 3A, electrodes 150 and 152 may be disposed on skin “s” adjacent wound “w” (e.g., on opposing sides of wound “w”). In this scenario, current flows from through the wound from one electrode positioned outside the wound to the other electrode positioned outside the wound. As best shown in FIG. 3B, electrodes 150 and 152 are disposed on an underside surface 142 of cover layer 134. Electrodes 150 and 152 may be thin metal, metallic paint, metallic foil, or any other suitable conductive material. Underside surface 142 may be composed of any high resistance material such as polythylene, poly(tetrafluoroethylene) (TEFLON™), polyurethane, polyester, a hydrogel made to be an insulator or any other suitable insulative material to prevent or greatly reduce the flow of current between electrodes 150 and 152. An adhesive on underside surface may provide a mechanism for affixing electrodes 150 and 152 to cover layer 134.

Wound dressing 120 also includes a conductive adhesive layer 116 contacting the underside of electrodes 150, 152. Adhesive layer 116 functions substantially as described above with respect to adhesive layer 16 and will not be discussed in further detail herein.

Wound dressing 120 also includes an autonomous power supply 180 that provides a voltage to electrodes 150 and 152 through electrical conductors 160 and 162, respectively. A resulting current flows from one electrode through the wound “w” to the other electrode to accelerate healing of wound “w”. The power supply 180 may be removably coupled to an upper surface of cover layer 134 via any suitable adhesive or mechanical connector (e.g., press-studs, grooves, hook-and-loop fasteners, etc.), as shown in the illustrated embodiment, or integrated into the wound dressing 120 and discarded with it after use. In embodiments, power supply 180 may include a battery (e.g., nickel cadmium, lithium-ion, alkaline, etc.) or any cell in which chemical energy is converted to electrical energy. The current and/or voltage supplied by power supply 180 may be fixed or it may be adjustable.

Although the foregoing disclosure has been described in some detail by way of illustration and example, for purposes of clarity or understanding, it will be obvious that certain changes and modifications may be practiced within the scope of the appended claims.

What is claimed is:

1. A negative wound pressure therapy apparatus comprising:
a wound dressing for defining a reservoir over a wound in which a negative pressure may be maintained by forming a substantially fluid-tight seal around wound tissue;
a fluid conduit in fluid communication with the reservoir and defining a fluid flow path for carrying fluids from the reservoir;
a vacuum source in fluid communication with the fluid conduit, the vacuum source suitable for providing an appropriate negative pressure to the reservoir to stimulate healing of the wound tissue; and
at least one biomedical electrode mounted with respect to the wound dressing for transmitting electrical energy to stimulate healing of the wound tissue.

2. The apparatus according to claim 1, further comprising a vacuum port adapted for connection to the fluid conduit, the vacuum port being coupled with the wound dressing to distribute negative pressure to the wound tissue.

3. The apparatus according to claim 2, wherein the vacuum port includes a connector, the connector adapted to couple with the at least one biomedical electrode to establish electrical communication therebetween.

4. The apparatus according to claim 3, wherein the connector is adapted to releasably couple with the at least one biomedical electrode.

5. The apparatus according to claim 2, wherein the vacuum port is integrally formed with the fluid conduit.

6. The apparatus according to claim 2, wherein the at least one biomedical electrode is disposed within the vacuum port.

7. The apparatus according to claim 1, further comprising an insulative material disposed between the at least one biomedical electrode.

8. The apparatus according to claim 1, wherein the wound dressing defines a flow path from the wound tissue to the fluid conduit for carrying fluids from the reservoir.

9. The apparatus according to claim 1, wherein a power source is operably coupled to the wound dressing.

10. The apparatus according to claim 1, wherein the fluid conduit is configured to electrically connect the at least one biomedical electrode to a power source.

11. The apparatus according to claim 1, wherein the wound dressing includes a conductive adhesive layer disposed about an underside of the at least one biomedical electrode and configured to adhere the at least one biomedical electrode to skin adjacent the wound tissue.

12. The apparatus according to claim 1, wherein the at least one biomedical electrode is at least a pair of biomedical electrodes mounted with respect to the wound dressing, the pair of biomedical electrodes configured to transmit electrical energy to stimulate healing of the wound tissue.

13. The apparatus according to claim 12, wherein the pair of biomedical electrodes are separated by an insulative material.

14. The apparatus according to claim 12, wherein the pair of biomedical electrodes are generally arcuate in shape to at least partially surround a flow path defined through the wound dressing, the flow path configured to carry fluids from the reservoir.

15. The apparatus according to claim 12, wherein one of the pair of biomedical electrodes is configured to transmit electrical energy through the wound tissue to the other biomedical electrode to stimulate healing of the wound tissue.
